(19) United States
(12) Patent Application Publication
(10) Pub. No.: US 2022/0236246 A1
(43) Pub. Date: Jul. 28, 2022

(54) PROCESS FOR SOURCE ATTRIBUTION

(71) Applicant: FONTERRA CO-OPERATIVE GROUP LIMITED, Auckland City (NZ)

(72) Inventors: Pierre VENTER, Auckland City (NZ); Grant Andrew ABERNETHY, Auckland City (NZ)

(21) Appl. No.: 17/595,902

(22) PCT Filed: May 27, 2020
(86) PCT No.: PCT/IB2020/054993

§ 371 Date: Nov. 29, 2021

(30) Foreign Application Priority Data
May 28, 2019 (AU) 2019901819

(51) Int. Cl. G01N 33/02; G06T 17/00; G16B 20/20; G16B 30/00

(52) U.S. Cl. CPC G01N 33/02; G06T 17/00; G06Q 50/12; G16B 30/00; G16B 20/20

(57) ABSTRACT

A computer implemented method for source attribution of a contaminant at a site. A computer implemented method for source attribution of a contaminant at a site and displaying a representation thereof. The method may be used to identify and/or manage contamination at a site, for example a food production site.

### FIELD OF THE INVENTION

The present invention relates to a computer implemented method for source attribution of a contaminant at a site. The present invention also relates to a computer implemented method for source attribution of a contaminant at a site and displaying a representation thereof. The method may be used to identify and/or manage contamination at a site, for example a food production site.

### BACKGROUND TO THE INVENTION

The identification and management of contamination is a challenge affecting many industries. For example, in the context of the food industry, contamination of consumable products has the potential for serious downstream effects on both the consumer and business involved. From a consumer perspective, food contamination may lead to consumer illness. From a business perspective, food contamination may have both a short and long-term negative impact. There may be an immediate or short-term impact related to loss of time and/or revenue associated with the allocation of resources to mitigate the contamination. Long term loss of revenue may also result from reduced consumer confidence in the quality and safety of the contaminated food product.

Methods of identifying and managing contamination have the potential to limit the impact of a contamination event on both consumers and businesses. Such methods may be applicable to a wide range of industries, such as for example, the food industry, forensics and the medical industry. Alternatively, such methods may target a specific industry or a subset of industries.

The efficient attribution of a contaminant to a source has the potential to further minimize the effect of a contamination event on both consumers and businesses. For example, in the context of the food industry, the efficient attribution of a contaminant to a source may prevent further instances of product contamination thereby allowing a business to prevent further instances of consumer illness.

There is a continuing need for methods of identifying a contaminant, attributing the source of a contaminant to at or near a surface within a site and/or managing a contamination event.

It is an object of the present invention to go some way to meeting this need and/or to at least provide the public and/or industry with a useful choice.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### SUMMARY OF THE INVENTION

In one aspect the present invention relates to a computer-implemented method for source attribution of a contaminant at a site, the method comprising

storing an electronic representation of the site in electronic memory, the representation comprising respective location information about one or more surfaces within the site,

receiving in the electronic memory contamination status information about a surface of the one or more surfaces,

modifying the representation to associate the location information with the contamination status information of the corresponding surface,

repeating the receiving and modifying steps to generate a data set comprising a plurality of associated contamination status information and location information, and

analysing the data set to attribute the source of the contaminant to at or near a surface of the one or more surfaces.

In another aspect the present invention relates to a computer-implemented method for source attribution of a contaminant at a site and displaying a representation thereof, the method comprising

generating, receiving or storing an electronic representation of the site in electronic memory, the representation comprising respective location information about one or more surfaces within the site,

generating or receiving in the electronic memory contamination status information about a surface of the one or more surfaces,

modifying the representation to associate or link the location information with the contamination status information of the corresponding surface, and

transmitting the modified representation to a display device for display to a user.

In various embodiments the method may comprise generating the representation by a method comprising

generating or receiving an initial representation of the site comprising a digital plan of the site, a 3D digital model of the site, or one or more images of the site, or any combination of any two or more thereof,

generating or receiving location information about respective one or more surfaces within the site,

storing the initial representation and the location information in a database, and

associating or linking the initial representation with the location information, such that a surface of the one or more surfaces can be identified by its respective location information.

In another aspect the present invention relates to a computer-implemented method for source attribution of a contaminant at a site and displaying a representation thereof, the method comprising

generating, receiving or storing in electronic memory respective location information about one or more surfaces within the site,

generating or receiving in the electronic memory contamination status information about a surface of the one or more surfaces,

transmitting the location information and the contamination status information to remote electronic memory comprising an electronic representation of the site,

receiving a modified electronic representation where the location information has been associated or linked with the contamination status information of the corresponding surface, and

displaying the modified electronic representation to a user.

The following embodiments may relate to any of the above aspects in any combination.

In various embodiments the contamination status information may comprise nucleic acid sequence information, may be generated according to a schedule, and may be determined by a method comprising collecting two or more samples from the one or more surfaces according to the schedule and analysing the two or more samples to determine the relatedness of the two or more samples

In various embodiments one or more of either or both of the generating steps and the transmitting step may be carried out using a point of use hardware device.

In various embodiments the contamination status information may be generated or received according to a schedule.

In various embodiments the contamination status information may comprise nucleic acid sequence information, amino acid sequence information, microbiological assay information, chemical assay information, or biochemical assay information, or any combination of any two or more thereof.

In various embodiments the nucleic acid sequence information may comprise one or more partial or whole genome sequences or mixed genome sequences.

In various embodiments the nucleic acid sequence information may comprise ribosomal RNA sequence information, single nucleotide polymorphism (SNPs) information or metagenomic information. In various embodiments the nucleic acid sequence information may comprise at least about 1, 2, 3, 4, 5, 6, 7, 8, 9, 10, 20, 30, 40, 50, 60, 70, 80, 90, 100, 250, 500, 750, or 1000 SNPs or more, and useful ranges may be selected between any of these values (for example, 1 to 5, 1 to 10, 1 to 50, 1 to 100, 1 to 1000, 5 to 10, 5 to 50, 5 to 100, 5 to 1000, 10 to 50, 10 to 100, or 10 to 1000).

In various embodiments the nucleic acid sequence information may be generated using nucleic acid sequencing methods including but not limited to Sanger sequencing, whole genome sequencing (WGS), or next generation sequencing (NGS).

In various embodiments the microbiological assay information may comprise mass spectrometry information.

In various embodiments the site may be a food production site, a food handling site, a food preparation site, a food logistics site, a food consumption site, an agricultural site, an animal handling site, a medical facility, a building, a vehicle, or a structure. The site may be a site where contamination must be controlled for protection of human or animal health. The site may be a site subject to a regulated hygiene standard, including but not limited to food code regulations, pharmaceutical manufacturing regulations, medical facility regulations, and the like.

In various embodiments the contamination status information may comprise information about a nucleic acid containing material, including but not limited to a bacteria, a virus, a protozoa, a plant, or an animal, or any combination of any two or more thereof.

In various embodiments the representation may comprise a 3D digital model, a point cloud, or one or more images of the site, or any combination thereof.

In various embodiments the point cloud may be generated by photogrammetrically processing the one or more images of the site.

In various embodiments the point cloud may be generated by laser, radar or sonar distance measurement of the site.

In various embodiments the representation may comprise unique identifiers linked to the one or more surfaces.

In various embodiments the contamination status information may be determined by a method comprising collecting one or more samples from the one or more surfaces and analysing the one or more samples. In various embodiments the one or more surfaces may include process equipment, ingredient, raw material, component, and packaging ingress points, process, packing and product egress points, cleaning equipment, hygiene control points, drains and other services, points of personnel ingress, movement, congregation, and egress, and personnel touch points including tools, handles and computer and control interaction points.

In various embodiments the one or more samples may be obtained according to a sampling plan.

In various embodiments the sampling plan may provide a first sampling location, a second sampling location, and an n^{th} sampling location, wherein each sampling location is determined by one or more statistically-based sampling methods.

In various embodiments the statistically-based sampling method may be simple random sampling, the method comprising selecting within the site a first random sampling location, a second random sampling location, and an n^{th} random sampling location.

In various embodiments the statistically-based sampling method may be systematic sampling, the method comprising defining a grid within the site, selecting a first sampling location on the grid, selecting a second sampling location on the grid, and selecting an n^{th} random location on the grid.

In various embodiments the statistically-based sampling method may be adaptive cluster sampling, the method comprising randomly selecting within the site a first set of primary sampling locations, determining whether each primary sampling location contains a contaminant, and selecting a second set of secondary sampling locations, each secondary sampling location being adjacent to a primary sampling location that contains a contaminant.

In various embodiments the one or more samples may be collected by swabbing, wiping, vacuuming, or blotting or any combination thereof.

In various embodiments the analysis of the one or more samples may comprise determining the presence of microbial contaminants using an assay to identify nucleic acid or amino acid information, or a microbiological assay, a chemical assay, or a biochemical assay, or any combination thereof.

In various embodiments analysis of the one or more samples may comprise determining the identity of a contaminant.

In various embodiments the analysis of the one or more samples may comprise determining the relatedness of two or more samples.

In various embodiments the analysis of the one or more samples may comprise determining the movement of a contaminant within a site by a method comprising

obtaining assay information from each of two or more samples obtained from different locations within the site,

determining the relatedness of the samples by analysing the assay information,

identifying potential vectors of contamination, and

determining the movement of the contaminant within the site by comparing the relatedness of the samples with the potential vectors.

In various embodiments the analysis of the one or more samples may comprise determining the identity of a microorganism contaminant by a method comprising

obtaining a first nucleic acid sequence from the one or more samples,

providing a second nucleic acid sequence from a reference microorganism,

defining a threshold of sequence similarity for establishing the identity of a microorganism in the one or more samples, and

determining whether the sequence similarity between the first and second nucleic acid sequences meets the threshold.

In various embodiments the threshold for establishing identity may be a sequence similarity of at least about 60, 70, 80, 90, 95, or 99 percent.

In various embodiments the analytical method may be repeated for 10, 100, or 100 or more nucleic acid sequences from the one or more samples.

In various embodiments the identity of the microorganism may comprise genus, species and/or strain information.

In various embodiments the method may comprise determining a level of risk associated with the microorganism contaminant by comparing the identity of the microorganism contaminant to a database comprising microorganism identifiers and associated risk or hazard information, and associating a risk or hazard to the microorganism contaminant.

In various embodiments the analysis of the one or more samples may comprise determining the relatedness of two or more samples by a method comprising

obtaining a first nucleic acid sequence from a first sample,

obtaining a second nucleic acid sequence from a second sample or from a reference source, and

comparing the first and second nucleic acid sequences to determine the relatedness of the samples.

In various embodiments comparing the first and second nucleic acid sequences to determine the relatedness of the samples may comprise

identifying one or more SNPs between each of the first and second nucleic acid sequences, and

comparing the SNPs from the first and second nucleic acid sequences to determine the relatedness of the samples.

In various embodiments the analysis of the one or more samples may comprise determining the movement of a microorganism contaminant within a site by a method comprising

obtaining nucleic acid sequence information from each of two or more samples obtained from different locations within the site,

determining the relatedness of the samples by analysing the nucleic acid sequence information,

identifying potential vectors of contamination, and

determining the movement of the microorganism contaminant within the site by comparing the relatedness of the samples with the potential vectors.

### Definitions

The phrase “machine-readable code” as used in this specification and claims is intended to mean, unless the context suggests otherwise, any form of visual or graphical code that represents or has embedded or encoded information such as a barcode whether a linear one-dimensional barcode or a matrix type two-dimensional barcode such as a Quick Response (QR) code, a three-dimensional code, or any other code that may be scanned, such as by image capture and processing.

The phrases “machine-readable medium” and “computer-readable medium” should be taken to include a single medium or multiple media. Examples of multiple media include a centralised or distributed database and/or associated caches. These multiple media store the one or more sets of machine or computer executable instructions. These phrases should also be taken to include any medium that is capable of storing, encoding or carrying a set of instructions for execution by a processor of a computing device and that cause the processor to perform any one or more of the methods described herein. The machine-readable medium or computer-readable medium is also capable of storing, encoding or carrying data structures used by or associated with these sets of instructions. These phrases include reference to solid-state memories, optical media and magnetic media.

The phrase “electronic memory” may include any local or remote machine readable medium, or combinations thereof, including cloud-based memory.

The term “comprising” as used in this specification means “consisting at least in part of”. When interpreting each statement in this specification and claims that includes the term “comprising”, features other than that or those prefaced by the term may also be present. Related terms such as “comprise” and “comprises” are to be interpreted in the same manner.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

As used herein the term “and/or” means “and” or “or”, or both.

As used herein “(s)” following a noun means the plural and/or singular forms of the noun.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

The invention consists in the foregoing and also envisages constructions of which the following gives examples only.

### BRIEF DESCRIPTION OF THE DRAWINGS

Preferred embodiments of the invention will be described by way of example only and with reference to the following drawings.

FIG. 1 is a flow chart showing steps in the method according to the first aspect of the invention.

FIG. 2 is a flow chart showing steps in the method according to the second aspect of the invention.

FIG. 3 is a flow chart showing steps in the method according to the third aspect of the invention.

### DETAILED DESCRIPTION

In the following description, specific details are given to provide a thorough understanding of the embodiments. However, it will be understood by one of ordinary skill in the art that the embodiments may be practiced without these specific details.

Also, it is noted that the embodiments may be described as a process that is depicted as a flowchart, a flow diagram, a structure diagram, or a block diagram. Although a flowchart may describe the operations as a sequential process, many of the operations can be performed in parallel or concurrently. In addition, the order of the operations may be rearranged. A process is terminated when its operations are completed. A process may correspond to a method, a function, a procedure, a subroutine, a subprogram, etc., in a computer program. When a process corresponds to a function, its termination corresponds to a return of the function to the calling function or a main function.

Aspects of the systems and methods described below may be operable on any type of general purpose computer system or computing device, including, but not limited to, a desktop, laptop, notebook, tablet, smart television, car audio or phone systems, game consoles or mobile device. The term “mobile device” includes, but is not limited to, a point of use hardware device, a wireless device, a mobile phone, a smart phone, a mobile communication device, a user communication device, personal digital assistant, mobile hand-held computer, a laptop computer, wearable electronic devices such as smart watches and head-mounted devices, an electronic book reader and reading devices capable of reading electronic contents and/or other types of mobile devices typically carried by individuals and/or having some form of communication capabilities (e.g., wireless, infrared, short-range radio, cellular etc.).

Furthermore, embodiments may be implemented by hardware, software, firmware, middleware, microcode, or any combination thereof. When implemented in software, firmware, middleware or microcode, the program code or code segments to perform the necessary tasks may be stored in a machine-readable medium such as a storage medium or other storage(s). A processor may perform the necessary tasks. A code segment may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

In the foregoing, a storage medium may represent one or more devices for storing data, including read-only memory (ROM), random access memory (RAM), magnetic disk storage mediums, optical storage mediums, flash memory devices and/or other machine-readable mediums for storing information. The terms “machine readable medium” and “computer readable medium” include but are not limited to portable or fixed storage devices, optical storage devices, and/or various other mediums capable of storing, containing or carrying instruction(s) and/or data.

The various illustrative logical blocks, modules, circuits, elements, and/or components described in connection with the examples disclosed herein may be implemented or performed with a general purpose processor, a digital signal processor (DSP), an application specific integrated circuit (ASIC), a field programmable gate array (FPGA) or other programmable logic component, discrete gate or transistor logic, discrete hardware components, or any combination thereof designed to perform the functions described herein. A general-purpose processor may be a microprocessor, but in the alternative, the processor may be any conventional processor, controller, microcontroller, circuit, and/or state machine. A processor may also be implemented as a combination of computing components, e.g., a combination of a DSP and a microprocessor, a number of microprocessors, one or more microprocessors in conjunction with a DSP core, or any other such configuration.

The methods or algorithms described in connection with the examples disclosed herein may be embodied directly in hardware, in a software module executable by a processor, or in a combination of both, in the form of processing unit, programming instructions, or other directions, and may be contained in a single device or distributed across multiple devices. A software module may reside in RAM memory, flash memory, ROM memory, EPROM memory, EEPROM memory, registers, hard disk, a removable disk, a CD-ROM, or any other form of storage medium known in the art. A storage medium may be coupled to the processor such that the processor can read information from, and write information to, the storage medium. In the alternative, the storage medium may be integral to the processor.

One or more of the components and functions illustrated the figures may be rearranged and/or combined into a single component or embodied in several components without departing from the invention. Additional elements or components may also be added without departing from the invention. Additionally, the features described herein may be implemented in software, hardware, as a business method, and/or combination thereof.

In its various aspects, the invention can be embodied in a computer-implemented process, a machine (such as an electronic device, or a general-purpose computer or other device that provides a platform on which computer programs can be executed), processes performed by these machines, or an article of manufacture. Such articles can include a computer program product or digital information product in which a computer readable storage medium containing computer program instructions or computer readable data stored thereon, and processes and machines that create and use these articles of manufacture.

The present invention broadly consists in a computer-implemented method for source attribution of a contaminant at a site. The methods described herein may be used for, for example, risk, issue or event management purposes. In various embodiments one or more of the methods described herein may provide an on-going risk assessment of a site of interest by identifying potential sources of contamination, including repeat sources of contamination, within a site.

In various embodiments the method comprises receiving contamination status information about a surface of the one or more surfaces within a site. In various embodiments the contamination status information may be generated or received according to a schedule (otherwise referred to as a sampling plan herein). Therefore, a method in accordance with the invention may provide a real-time method of monitoring one or more contaminants at a site such that the method may form part of a regular contaminant monitoring program. In various embodiments the schedule may specify at least 2, 3, 4, 5, 6, 7, 8, 9, 10, 20, 30, 40, 50, or 100 or more sample collections to generate contamination status information about the one or more surfaces, and useful ranges may be selected between any of these values (for example, 2 to 7, 2 to 10, 2 to 30, 2 to 50, or 2 to 100). In various embodiments the schedule may specify at least 2, 3, 4, 5, 6, 7, 8, 9, 10, 20, 30, 40, 50, 100, 250, 500, 750, or 1000 or more surfaces (sampling locations) for which to generate contamination status information, and useful ranges may be selected between any of these values (for example, 2 to 7, 2 to 10, 2 to 30, 2 to 50, or 2 to 100). In various embodiments the schedule may relate to a time period of at least about 1, 2, 3, 4, 5, 6, 7, 8, 9, 10, 11, 12, 13, 14, 15, 20, 30, 40, 50, 60, 70, 80, 90, or 100 days, and useful ranges may be selected between any of these values (for example, 1 to 7, 1 to 14, 1 to 30, 1 to 60, 1 to 90, 7 to 14, 7 to 30, 7 to 60, or 7 to 90).

In various embodiments the method may be used in the source attribution of one or more contaminants within a site. The contaminant may be any undesirable or hazardous substance or organism, such as a polluting, poisonous, or toxic substance or organism. Such substances may include any substance that is undesirable or unacceptable in the context of the site, such as heavy metals, antibiotics, toxins, pesticides or herbicides at a food preparation site. Such organisms may include any organism that is undesirable or unacceptable in the context of the site, such as spoilage organisms or organisms presenting a food safety risk at a food preparation site. Alternatively or additionally, the contaminant may be any substance or organism of interest at a site, where the identity and/or source of the substance or organism at the site is of interest. For example, a nucleic acid sequence of interest. The contaminant may be a single substance or organism or a mixed population of substances or organisms.

The contaminant may be industrial, agricultural, chemical or biological in nature. Examples of industrial contaminants may include but are not limited to volatile organic compounds (VOCs) and heavy metals. Examples of agricultural contaminants may include but are not limited to pesticides such as insecticides, herbicides and/or fungicides. Examples of chemical contaminants may include but are not limited to solvents and organic and/or inorganic chemical waste products. Examples of biological contaminants may include any nucleic acid containing material, including but not limited to microorganisms.

In various embodiments the contaminant may be a bacterium, virus, protozoa, fungi, plant or animal, or a combination of any two or more thereof. Bacterial contaminants may include any bacterium of interest, including for example, food spoilage bacteria such as Lactobacillus spp., Leuconostoc spp., Pseudomonas spp., Alcaligenes spp., Serratia spp., Micrococcus spp., Flavobacterium spp., Serratia spp., Micrococcus spp., Proteus spp., Enterobacter spp., Streptococcus spp. or any combination of any two or more thereof, and/or pathogenic bacteria such as Campylobacter jejuni, Escherichia coli, Listeria monocytogenes, Salmonella spp., Shigella spp., Staphylococcus aureus, or any combination of any two or more thereof. Fungal contaminants may include any fungi of interest, including for example, food spoilage fungi such as Aspergillus spp., Fusarium spp., Cladosporium spp., Alternaria spp. or any combination of any two or more thereof. Animal contaminants may comprise, for example vermin or vectors. Vermin or vectors may comprise, for example, rodents such as mice or rats; insects such as cockroaches or mosquitos; parasites such fleas, ticks, bed-bugs, lice or termites; or any combination of any two or more thereof.

In various embodiments the contaminant is a microbial contaminant, for example a bacterium or a virus, and in various embodiments the method described herein may be used in the source attribution of the microbial contaminant at a site. In such embodiments, the contamination status information may comprise nucleic acid sequence information, amino acid sequence information, microbiological assay information, chemical assay information, or biochemical assay information, or any combination of any two or more thereof.

In various embodiments the contaminant is or comprises one or more nucleic acid sequences of interest, and the one or more nucleic acid sequences or the nucleic acid information may include for example, an oligonucleotide, a polynucleotide, a transposon, a contiguous nucleic acid of 10, 100, or 1000 or more bases, one or more DNA molecules, one or more RNA molecules, DNA from one or more individual genes, one or more partial or whole genome sequences, one or more partial or whole RNA transcriptomes, or one or more ribosomal RNA sequences, or other information related to any such sequences.

The method described herein may be used for the source attribution of only one contaminant within a site. Alternatively, in various embodiments the method described herein may be used for the source attribution of more than one contaminant within a site. In such embodiments each of the contaminants may be the same type of contaminant selected from the group consisting of a bacterium, virus, protozoa, plant and animal. For example, the method may be used for the source attribution of two contaminants, both contaminants being bacteria. In various embodiments the method may be used for the source attribution of different types of contaminants. For example, one of the contaminants may be a bacterium and the other may be a virus. Additionally or alternatively, the contaminant may be a mixed population of contaminants, such as a mixed population of bacteria.

In various embodiments the method comprises collecting a sample from a surface of a site and analysing the sample to determine the presence, absence or amount of one or more contaminants in the sample.

In various embodiments sample analysis may comprise several levels of analysis. A first or high-level of analysis may comprise one or more methods of determining whether one or more contaminants are present on a surface. For example, if the contaminant is a bacterium then a first or high-level analysis may comprise plated media tests or polymerase chain reaction (PCR) based methods. In various embodiments samples that are positive for contaminants may be screened to select samples for subsequent analysis or analyses as described herein. In various embodiments samples may be analysed for suitability or need for subsequent analysis for example by determining the identity of the contaminant, determining the distribution of the contaminant, and/or determining the level of risk or hazard posed by the contaminant. Samples may be selected based on one or more selection criteria.

Subsequent analysis or analyses may comprise one or more methods of determining the nature of the contaminant present. That is, subsequent analyses may provide additional information about the particular contaminant(s) that is/are present.

In various embodiments sample analysis may comprise the use of one or more tests for determining specific information about a contaminant. The test may be carried out by a sensor, where practical.

As described herein, in various embodiments sample analysis may comprise determining the identity of a contaminant, determining the relatedness of two or more samples, and/or determining the movement of a contaminant within a site.

In various embodiments specific information about a contaminant may comprise chemical assay information or biochemical assay information generated in relation to a single analyte or multiple analytes that comprise or are comprised within a sample. The assay may comprise a binding assay, immunoassay, colourimetry assay, photometry assay, spectrophotometry assay, transmittance assay, turbidimetry assay, counting assay, flow cytometry assay, imaging assay, enzyme-linked immunoassay (EIA), enzyme linked immunosorbent assay (ELISA), microarray assay, enzyme assay, or mass spectrometry assay, or any combination of any two or more thereof. Such assay information may be used to determine the identity of a contaminant, and comparison of such assay information between samples may be used to determine the relatedness of two or more samples, and/or determine the movement of a contaminant within a site. Similar assay results can indicate a degree of relatedness. It should be appreciated that any suitable assay may be used.

In various embodiments specific information about a contaminant may comprise nucleic acid sequence information, for example, an oligonucleotide, a polynucleotide, a transposon, a contiguous nucleic acid of 10, 100, or 1000 or more bases, one or more DNA molecules, one or more RNA molecules, DNA from one or more individual genes, one or more partial or whole genome sequences, one or more partial or whole RNA transcriptomes, or one or more ribosomal RNA sequences. In various embodiments the nucleic acid sequence information may comprise one or more genes, one or more non-coding regions, or one or more fragments of a nucleic acid sequence, or other information related to any such sequences.

In various embodiments specific information about a contaminant may comprise microbiological assay information. The microbiological assay information may comprise, for example, information about a single organism or a mixed population of organisms, including information generated by one or more assays including but not limited to a binding assay, immunoassay, colony forming assay, culture assay, colourimetry assay, photometry assay, spectrophotometry assay, transmittance assay, turbidimetry assay, counting assay, flow cytometry assay, imaging assay, enzyme-linked immunoassay (EIA), enzyme linked immunosorbent assay (ELISA), microarray assay, enzyme assay, polymerase chain reaction (PCR) assay, or mass spectrometry assay, or any combination of any two or more thereof. Mass spectrometry may include Matrix Assisted Laser Desorption/Ionization Time-of-flight Mass Spectrometry (MALDI-TOF) information. For example, if the contaminant is a microorganism such as a bacterium, a Bruker BioTyper may be used to confirm the type of bacterium present, for example the genus and/or species of bacterium.

In various embodiments samples may be analysed by whole genome sequencing (WGS) or next generation sequencing (NGS). In various embodiments advanced tests, such as WGS, may be used as part of subsequent analyses performed on a sample. That is, advanced tests may be performed after a first or high-level tests. In various embodiments metagenomic methods such as shotgun metagenomic sequencing may be used to analyse samples comprising heterogenous populations of microbial contaminants or samples comprising microbial contaminants that cannot be cultured using traditional methods.

Advanced tests, for example WGS or NGS, may allow hypotheses or conclusions to be made about the relationships between contaminants in a sample. For example, WGS, preferably in combination with statistical analyses, may allow a connection between microorganisms in two or more samples, for example samples collected from different surfaces, to be made. Such connections could lead to a hypothesis or conclusion that the microorganisms
from the two or more samples are from the same population,
from the two or more samples evolved or originated from the same population (the “original population”), or that
from one of the two samples evolved or originated from another of the two samples.

In various embodiments the method described herein associates the location information and the contamination status information of a corresponding surface. As such, in various embodiments the method described herein may be used to attribute the source of a contaminant such as a microorganism collected from a first surface A to a second surface B. In such embodiments, the second surface B may comprise the original source or population.

Whole genome sequencing may be carried out locally at the site, or remotely and the contamination status information transmitted to and received at the site. WGS may be carried out using a combination of software and associated hardware. For example, WGS may be carried out using polymerase chain reaction (PCR), gel electrophoresis, pulsed field gel electrophoresis (PFGE), or next generation sequencing using an Illumina MiSeq or related high-throughput genetic analysers with associated software and kit-based workflows. Other instruments and protocols for generating genome sequence data may also be used including for example, ABI Sanger chemistry-based sequencer, next generation sequencers such as Pacific Biosciences PacBio single molecule real time (SMRT) sequencer, Ion Torrent semiconductor sequencer, Oxford Nanopore MinION, and other high-throughput approaches that will be apparent to a person skilled in the art. WGS may be carried out using any one or any combination of any two or more such techniques.

Bioinformatic-based approaches may be used in conjunction with sequencing methods such as WGS or NGS to analyse nucleic acid sequence information. For example, bioinformatic-based approaches may be used to compare nucleic acid sequence information to determine identity of microbial contaminants, relatedness of different samples of microbial contaminants, mapping movement of microbial contaminants within a site, and/or determining the source of microbial contaminants. Bioinformatic approaches may be carried out using personalized statistical software, or proprietary software. Software may be local machine based software for example Illumina MiSeq integrated applications or internet based tools, for example, sequence alignment software such as basic local alignment search tool (BLAST), Illumina BaseSpace Sequence Hub Apps, or access to internet based databases such as National Center for Biotechnology Information (NCBI), or specific gene or genome databases, or any combination thereof.

In various embodiments the nucleic acid information may be processed to obtain sequence data of suitable quality for further analysis. For example, the sequence data processing may comprise demultiplexing, trimming, removal of low-quality sequences, removal of noise, error detection, assembly alignments, and statistical quality control and other techniques that will be apparent to a person skilled in the art. Sequence data processing may be carried out using personalized statistical software based on Perl, R, Python, C, Java, or similar programming environments, open license applications for example Trimmomatic (usadellab.org), or proprietary, local software such as Illumina MiSeq integrated applications or internet based solutions such as Illumina BaseSpace Sequence Hub Apps.

In various embodiments the identity of the microbial contaminant(s) may be determined by comparing the processed genome sequence data against known reference sequences. The reference sequence may be whole or partial genome sequence of microorganisms of interest stored in an electronic database for example, NCBI, EMBL or GenBank. In various embodiments the identity comprises the genus, species and/or strain of the microbial contaminants.

In various embodiments identification of microbial contaminants isolated from a location in a site may comprise determining sequence similarity or homology between the processed nucleic acid sequence information (target sequence) and one or more reference sequences. Such determination may be carried out using methods known in the art. Such methods may include defining consistent length, overlapping short sequences (k-mers) derived from the target sequence, associating each k-mer with sequence and taxonomic information in a reference database, and identifying the target sequence and organism using a weighting algorithm based on the numbers of k-mers associated with each taxonomic unit in the reference database. Determination of sequence similarity may be carried out using internet based software for example Kraken (John Hopkins University Centre for Computational Biology). In various embodiments the threshold for establishing identity of a microbial contaminant may be a sequence similarity of about 60, 70, 80, 90, 95, or 99 percent, and suitable ranges may be selected from any one of these values, for example about 60 to 99, 60 to 95, 60 to 90, 60 to 80, 60 to 70, 70 to 99, 70 to 95, 70 to 90, 70 to 80, 80 to 99, 80 to 95, 80 to 90, 90 to 99 or 95 to 99 percent.

In various embodiments Sanger-based methods or alternative long-read next generation WGS methods, for example PacBio SMRT sequencing, may be used to create a sample and/or reference sequence without prior knowledge.

The identity such as the genus and species of the microbial contaminant may be used to determine the hazard and/or level of risk posed by the microbial contaminant. In various embodiments the method comprises establishing identity of the microbial contaminant, comparing the identity of the microbial contaminant to a database of microorganism identifiers and associated risk or hazard information, and associating a risk or hazard information to the microbial contaminant. Risk or hazard information may comprise safety based criteria such as hygiene, pathogenicity or virulence, or business based criteria such as quality or regulatory considerations.

Variations in the nucleic acid information such as Single Nucleotide Polymorphisms (SNPs) may be used to determine relatedness of a sample of microbial contaminant with a known reference strain or other samples of microbial contaminant. SNPs may occur anywhere in a genome, for example, within gene(s) or intergenic region(s). Calculation of the SNP value, or count of SNPs, of the nucleic acid sequence information may indicate that the sample of microbial contaminant is related to a reference strain or other sample microbial contaminant.

In various embodiments the relatedness of a sample of microbial contaminant and a reference strain may be determined by identifying SNPs in the nucleic acid sequence information of a microbial contaminant, compared to the nucleic acid sequence information of a reference strain, and determining the level of similarity of those sequences based on the number of SNPs, where a value of zero indicates identical strains, and higher SNP values indicates some degree of non-similarity between sequences and thus strains.

In various embodiments the determination of relatedness of two or more samples of microbial contaminants may comprise identifying SNPs between a first nucleic acid sequence information of a first microbial contaminant, and a second nucleic acid sequence information of a second microbial contaminant and determining similarity between the first and second nucleic acid sequence information based on the count of SNPs. For example, small differences between the SNPs of the first nucleic acid sequence information and the second nucleic acid sequence information may indicate that the microbial contaminants are closely related. In various embodiments relatedness of a microbial contaminant and a reference strain may be determined. The identification of SNPs may be carried out using software for example SNIPPY (GitHub.com/tseemann).

Where microbial contaminants are determined to be related, comparison of the SNPs of these microbial contaminants may be used to determine the length of time since microbial contaminant populations diverged/evolved from a common ancestor population (relative age of each population). Depending on the microorganism, it may be possible to determine the number of weeks, months or years since a microbial contaminant diverged/evolved from a common ancestor. In various embodiments determination of the length of time it takes for a given microbial contaminant population(s) to evolve from a common ancestor may be calculated by determining the rate of change of SNPs for the microorganism of interest.

Expression of genes may result in specific traits in a microbial contaminant. These traits may be relevant to the survivability of the microbial contaminant at specific locations within a site. In various embodiments information on these traits may be used to attribute a microbial contaminant population displaying specific traits to corresponding specific locations within a site. For example, a salt tolerance trait in a microbial contaminant population may be used to attribute that microbial contaminant, or its ancestor, to a location that is exposed to higher salt content.

In various embodiments transmission of specific traits in populations of microbial contaminants may be used to determine relatedness between the populations. In various embodiments the method may comprise identifying a trait or phenotype in a microbial contaminant, attributing the trait or phenotype to a gene or genes in the nucleic acid sequence information of the microbial contaminant, identifying the presence of the gene in the nucleic acid sequence of other samples of microbial contaminants, determining relatedness based on the presence of the gene in the other samples of microbial contaminants.

Variation in specific gene(s) or specific region(s) within the genome of microbial contaminants may also be used to determine relatedness. For example, genes such as ribosomal genes may comprise slowly evolving conserved regions and/or fast evolving regions. The slow evolving conserved regions of such a gene may be used to determine longer period diversions from an ancestor, such as genus and/or species or higher level taxonomic level identifications, while the fast-evolving regions may be used to identify specific strains within species.

The movement of microbial contaminants within a site may be mapped by identifying changes in the SNPs from two or more populations of microbial contaminants and the locations within a site from which each population was isolated with time. As a microbial contaminant population (original population) is transferred from a first location to a second location (for example by a vector), the SNPs of the transferred (second) microbial contaminant population may change in time relative to the founder population. If the second population is transferred to a third location, the SNPs of the third microbial contaminant population may change further. This results in a sequence of microbial contaminant populations each with a varying degree of change in SNPs relative to the original population. In other words, the movement of the microbial contaminant may be mapped by determining the change in SNPs of the population and relating it back to the locations each population was isolated over time. Vectors in this context may include, for example, animals (including humans and pest species), incoming goods (including manufacturing ingredients or components and cleaning products), waste streams, water, air, tools (including cleaning tools), and vehicles.

In various embodiments the method of mapping the movement of microbial contamination comprises identifying SNPs in the genetic sequences of two or more microbial contaminant populations, each isolated from different locations within a site, forming a sequence of microbial contaminant populations by arranging each population according to similarity by SNPs from most similar to most dissimilar, and mapping movement of a microbial contaminant by attributing each microbial contamination population to the location within the site it was isolated. For example, five samples of microbial contaminants isolated from locations A, B, C, D and E when arranged according to degree of similarity in SNPs may result in the order C, E, B, D, A. It may be possible to infer that the microbial contaminant moved over time from location C to E to B to D to A or vice versa.

Alternatively or additionally, by comparing the degree of change in SNPs of multiple samples of microbial contaminants to a known reference strain, it may be possible to identify the sample that comprises the original microbial contaminant population in a site. In various embodiments the method may comprise comparing identifying the SNPs in two or more samples of microbial contaminants, comparing the SNPs in each sample of microbial contaminant with the SNPs in a reference strain, determining the degree of change of SNPs in each sample compared to the reference strain, identifying the sample with the smallest change in SNPs and attributing that sample as the original population.

In various embodiments, SNP analysis useful herein may comprise comparing a sample genome sequence to a reference genome of the same species and recording the differences. The differences may be use used to determine if the sample contaminant is comprised of a single strain or multiple strains, a persistent strain or transient strains, and if the sample contaminant is the result of a single incursion, multiple incursions, or is endemic in the site. A known molecular evolutionary time period for SNP changes within the species of interest may be used to determine relatedness and/or clonality.

In various embodiments the method may comprise multiple rounds of mapping the movements of microbial contamination populations as described above, for example two or more, three or more, four or more, five or more, six or more, seven or more, eight or more, nine or more, 10 or more rounds. In various embodiments different microbial contaminant samples may be used in each round. The results from multiple rounds of mapping, over time, may be combined to increase the accuracy or resolution of the map.

Epidemiological approaches may be used in conjunction with or in addition to bioinformatic approaches to increase the accuracy of source attribution. The similarly of samples as established by various embodiments may imply that two or more contaminations are related. Epidemiological considerations are also required to give context to the relationship between contaminations, to establish that movement between the samples locations was possible, and to identify vector(s). Consideration of likely vectors, for example the scheduling or movement of people, equipment, ingredients, product, cleaning agents, packaging, or movement of water, waste, air or other services, or incidental events for example breakdowns, maintenance or vermin within a site may be used help attribute the source of contamination. Similarity, considerations of solid barriers to movement, for example in construction, scheduling, or dismissal of low risk events can add weight to the conclusions that help attribute the source of contamination.

Accordingly, in various embodiments sample analysis may comprise determining the movement of a contaminant within a site by a method comprising obtaining assay information from each of two or more samples obtained from different locations within the site, determining the relatedness of the samples by analysing the assay information, identifying potential vectors of contamination, and determining the movement of the contaminant within the site by comparing the relatedness of the samples with the potential vectors.

Statistical approaches may be used to assess the confidence in the results obtained. In various embodiments statistical methods may be used to test for non-randomness of the results. In various embodiments spatial statistics may be used to distribution of microbial contaminant populations are non-random. For example, statistical methods may be used to calculate the probability that the spread of contaminant populations around a site was non-random.

It will be understood by a person skilled in the art that carrying out different levels of sample analysis may be cost effective for a business. For example, a first or high-level analysis may comprise a relatively cheap method of determining whether one or more contaminants are present. If the first analysis does not identify any contaminants on the surface tested, then the business does not need to spend additional time and money on further testing. If the first analysis identifies contaminants on the surface tested, then a decision can be made about whether to carry out more targeted analyses to determine the nature, for example amount, of contaminant present.

In various embodiments one or more samples may be taken from a surface without prior knowledge of the potential contaminants that may be found on the surface. In various embodiments, for example in cases of suspected contamination, the company, co-operative or individual carrying out a method in accordance with the invention may carry out the method to test for the presence of a particular contaminant or contaminants. It will be apparent to a person skilled in the art that the type of analysis carried out to determine contamination status information of a surface will depend at least in part on whether the nature of the contaminant is known.

The site may comprise one or more indoor and/or outdoor surfaces. In various embodiments the site may be a site such as those discussed above.

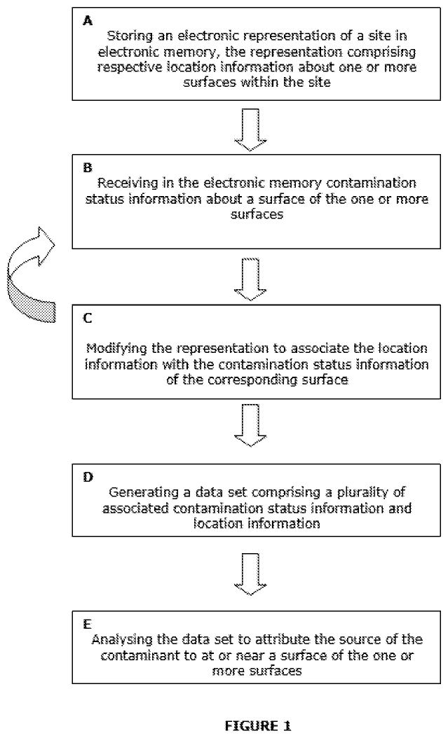

The invention will now be described in more detail with reference to the accompanying figures in which FIG. 1 shows a method for source attribution of a contaminant at a site, the method comprising
A. storing an electronic representation of the site in electronic memory, the representation comprising respective location information about one or more surfaces within the site,
B. receiving in the electronic memory contamination status information about a surface of the one or more surfaces,
C. modifying the representation to associate the location information with the contamination status information of the corresponding surface,
D. repeating the receiving and modifying steps to generate a data set comprising a plurality of associated contamination status information and location information, and
E. analysing the data set to attribute the source of the contaminant to at or near a surface of the one or more surfaces.

With continued reference to FIG. 1, step A comprises storing an electronic representation of a site in electronic memory. In various embodiments the electronic memory may be stored on a storage medium as described herein.

The electronic representation may comprise a plan of the site, map of the site and/or a collection of photographs or images of the site. In various embodiments the electronic representation comprises a map of the site and the electronic representation is created by mapping a site. The map may be 2-dimensional (2D), 3-dimensional (3D) or 4-dimensional (4D). Mapping a site may comprise the use of a non-digital map which is then digitized to create an electronic representation of the site. In various embodiments digital images of a site may be used to create an electronic representation of the site. In various embodiments both digital and non-digital images may be combined to create an electronic representation of the site. In various embodiments the map or plan of the site may be created using any suitable computer-based method, for example, computer-aided design (CAD). In various embodiments the map or plan of the site may be created using suitable surveying tools, for example, theodolite and measuring tapes.

Methods of mapping a site may be selected based, at least in part, on factors such as for example whether the site is an indoor or outdoor site. It will be understood by a person skilled in the art that some methods of mapping may be applicable to both internal and external sites while others may be limited to either internal or external sites. As examples, in various embodiments external sites may be mapped using drones, aircraft-based sensors, other aerial sensors, or satellite-based sensors. In various embodiments drones and other aerial sensors may also be used to map indoor sites, in particular, large indoor sites such as warehouses. Mapping may also be based on computer-aided design (CAD) models of a site.

In various embodiments a site, for example an indoor site or indoor area or surfaces of a site, may be mapped using distance scanning technologies, such as those employing electromagnetic radiation (EMR) or sound, including laser, radar, or sonic scanning technologies. Accordingly, in various embodiments a representation of a site, such as a point cloud, stick model, vector model, or 3D digital model may be generated by EMR or sonic measurement of a site, such as by light detection and ranging (LIDAR). Suitable distance scanning technologies are known in the art, such as laser scanners including, for example, FARO® Focus laser scanners from FARO® Technologies UK Ltd. Typically, distance scanners scan a site to be mapped by emitting pulses of light, radio or sound and measuring the ‘time of flight’, or the time it takes for the signal to be reflected back to the scanner. In various embodiments the time of flight measurement may be combined with other information such as the angle of each signal to obtain a data point (or a coordinate) in a point cloud, stick model, or vector model. Such scanners may take at least about 1, 10, 20, 30, 40, 50, 100, 150, 200, 250, 300, 350, 400, 450, 500, 550, 600, 650, 700, 750, 800, 850, 900, 950, 1000 scans or more of a particular site, and suitable ranges may be selected from any one of these values, for example from about 1 to 10, 10 to 1000, 10 to 750, 10 to 500, 10 to 350, 10 to 250, 10 to 100, 10 to 500, 100 to 1000, 100 to 750, 100 to 500, 100 to 350, 100 to 250, 200 to 1000, 200 to 750, 200 to 500, 200 to 350, 250 to 1000, 250 to 750, 250 to 500, 250 to 350, 500 to 1000 or 500 to 750 scans.

In various embodiments the distance scanning technologies may be used to generate a single point cloud image or model of the site. Point cloud images generated by scanning technologies, for example 3D laser scanning technologies, are used to generate point cloud images or models referred to herein as point clouds, internal point cloud images or models. The point cloud image or model is made up of the data points within the site. The data points from two or more cloud point images may be combined together using computer software, such as, for example FARO® Scene. In various embodiments the point cloud image or model may comprise millions or billions of points per cloud. Stick and vector models may be generated and manipulated in similar ways, known in the art.

In various embodiments the distance scanning technologies may be used to generate more than one point cloud image or model of the site. For example, several point cloud images or models may be generated, each of the images or models corresponding to a particular section of the site, for example a particular surface, group of surfaces, or room within the site. Stick and vector models may be generated and manipulated in similar ways, known in the art.

Each scan taken by a laser scanner, for example FARO® Focus laser scanner, may comprise millions of laser distance measurements. Such laser distance measurements may be panoramic. In various embodiments the laser distance measurements may be combined with one or more photographs or videos, for example at least about 1, 2, 3, 4, 5, 6, 7, 8, 9, 10, 11, 12, 13, 14, 15, 20, 25, 50, 100, 125, 150, 200, 300, 400, 500, 600, 700, 800, 900, 1000 or more photographs or videos, preferably photographs, to create an electronic representation of a site. In various embodiments, the electronic representation of the site may be panoramic.

In various embodiments one or more photographs and/or videos of a site may be combined to create the electronic representation. Accordingly, in various embodiments the point cloud may be generated by photogrammetrically processing one or more images of a site.

In various embodiments one or more photographs and/or videos of a site may be combined with one or more distance scans, such as scans taken by a laser scanner, for example FARO® Focus laser scanner, to create the electronic representation. Accordingly, in various embodiments the electronic representation may comprise a point cloud and one or more images of a site.

The one or more photographs and/or videos may be captured using, for example drones; aircraft-based sensors; other sensors, including for example other aerial sensors, satellite-based sensors; or cameras such as, for example, hand-held, backpack, trolley or head mounted, vehicle or robot-mounted cameras; or a combination of any two or more thereof.

Photographs and/or videos used to create the electronic representation may be black-and-white and/or colour photographs. In various embodiments the photographs and/or videos, preferably photographs, may be embedded into the electronic representation, for example, using links. Alternatively, or additionally, in various embodiments the photographs and/or videos may be published as a web-share companion to the scans from the laser scanner.

In various embodiments one or more photographs may be converted into one or more external point cloud, stick or vector models. Software for converting one or more photographs into such models is known in the art and includes but is not limited to Bentley photogrammetric imagery software.

In various embodiments one or more external such cloud models and one or more internal such cloud models may be combined to create the electronic representation of a site. The representation may also include data transformations to other digital formats, such as conversion of point clouds to vector models by Pointfuse software, or reformatting to solid surface models, suitable for computer aided drafting (CAD) processing.

In various embodiments the method described herein comprises collection of one or more samples from one or more surfaces of a site. Each surface of the one or more surfaces is located at a specific position in the site. As described herein the method may comprise generating, receiving or storing in electronic memory an electronic representation of a site, the representation comprising respective location information about one or more surfaces within the site. That is, the one or more surfaces are identifiable by their location on the representation. Therefore, in various embodiments collection of a sample from a surface of the one or more surfaces also comprises collection of information about the location of the surface from which the sample was collected within the site.

In various embodiments the location of surfaces in a site from which samples are collected (sampling locations) may be determined by a sampling plan. In various embodiments the sampling plan defines the time and/or location at which samples are collected. In various embodiments, the sampling plan may comprise determining a first sampling location, determining a second sampling location, and determining n^{th} sampling locations on an electronic representation of the site for a given time. 1, 2, 4, 6, 8, 10, 20, 30, 40, 50, 100, 150, 200, 400, 600, 800, or 1000 or more sampling locations may be chosen by this method, and useful ranges may be chosen between these values (for example 1 to 150 sampling locations).

In various embodiments each sampling location may be determined by statistically-based sampling methods for example, simple random sampling, systematic sampling, ranked set sampling, adaptive cluster sampling or a combination of two or more thereof. In various embodiments each sampling location may be determined by judgment-based sampling methods for example, using expert or industry knowledge. Expert or industry knowledge may comprise for example, knowledge on locations within a site that has an increase probability of being contaminated. In various embodiments statistically-based methods is supplemented by judgment-based sampling methods to improve accuracy of the statistically-based method.

Location information about the one or more surfaces in a site may be collected in a number of ways. In various embodiments location information may be collected manually or automatically.

In various embodiments location information may be collected manually by a person collecting the one or more samples from a surface, or by a different person. For example, the person collecting the location information may mark or otherwise record the location of the surface from which a sample is collected on a non-digital map. The non-digital map may then be digitized to create an electronic representation of the site as described herein. Alternatively, the person collecting the location information may mark or otherwise record the location of the surface from which a sample is collected on a digital map. That is, the method may comprise generating, receiving or storing an electronic representation of a site in electronic memory and a person collecting a sample from a surface may mark or otherwise record the location of the surface from which the sample was collected on the electronic representation.

In various embodiments location information may be collected automatically. For example, location information may be collected using a local or global navigation satellite system (GNSS) such as global positioning system (GPS).

The respective location information may be displayed on the representation in several ways. For example, a reference numeral may be assigned to each of the one or more surfaces. The reference numerals may then be displayed on the representation to indicate the location of each of the one or more surfaces within the site. Alternatively, or additionally, the respective location information may be shown by, for example, assigning a colour and/or a symbol to each of the one or more surfaces. The location of each one of the surfaces may then be identifiable by the colour and/or the symbol on the representation.

In various embodiments the respective location information about one or more surfaces within the site may be displayed using a shape corresponding to the surface. The shape may be a 2D or 3D shape. The shape may be a line, an arrow, a pointer, a label, text, a hyperlink, an image or any symbol. For example, in various embodiments the electronic representation may comprise 3D spheres, each 3D sphere corresponding to a surface within the site. The shape, for example the 3D sphere, may have a size that is proportional to the size of the surface. Alternatively, the shape, for example the 3D sphere, may have a size that does not correlate with the size of the surface. That is, the size of the shape may serve only to indicate the respective location of the surface. Software for displaying shapes on electronic representations is known in the art, and includes but is not limited to, for example, Veesus, FARO® Scene and AutoCAD software.

The electronic representation may comprise data other than the respective location information or contamination status information about one or more surfaces of a site. In various embodiments the electronic representation may comprise or be used to display metadata. Metadata is used herein to refer to data other than location information or contamination status information about one or more surfaces of a site.

Metadata may be displayed on the electronic representation in several ways. For example, metadata may be displayed on the electronic representation using for example, colours, symbols and/or shapes, text or hyperlinks corresponding to different types of metadata. Alternatively, or additionally, metadata may be tagged to or otherwise associated with the respective location information about one or more surfaces in the electronic representation. For example, metadata may be tagged to or otherwise associated with the respective location information about one or more surfaces in the electronic representation using a unique identifier.

In various embodiments a unique identifier may be associated with all or some data corresponding to a surface of the one or more surfaces within a site. In various embodiments the electronic representation may comprise one or more unique identifiers linked to the one or more surfaces of a site. The unique identifier may comprise respective location information, contamination status information and/or metadata about a surface of the one or more surfaces within a site. The unique identifier may be associated with sample labels and testing allowing data to be correctly correlated with the location corresponding to the surface tested. The unique identifier may be or may be associated with a machine-readable code.

Metadata may be data relating to the nature, for example shape, volume, area or appearance, of the surface from which a sample was collected; information indicating the time and/or date that the sample was collected; information identifying the individual responsible for collecting the sample; information relating to the type of sample collected from the surface; information relating to the method of collecting the sample from the surface; information prescribing an instruction following collection of the sample; information about conditions at the site at the time that the sample was collected, for example temperature, humidity and/or pH level; or a combination of any two or more thereof. Other types of metadata will also be apparent to a person skilled in the art.

As described above, step B of the method of FIG. 1 comprises receiving in the electronic memory contamination status information about a surface of the one or more surfaces. In various embodiments receiving in the electronic memory contamination status information comprises collecting such information from a surface of the one or more surfaces. Such information may be collected by collecting one or more samples from the surface of the one or more surfaces.

It will be understood by a person skilled in the art that contamination status information may be collected in many ways. The method of collecting the contamination information may depend on factors such as the type of contamination under assessment and/or the site being assessed. For example, the contamination status information may be collected by collecting one or more samples from a surface of the one or more surfaces of a site. Samples may be collected, for example by swabbing, wiping, vacuuming, or blotting. Surfaces may be sampled manually or automatically.

In various embodiments multiple samples may be collected from each surface of the one or more surfaces of a site. For example, at least about 1, 2, 3, 4, 5, 6, 7, 8, 9, 10, 15, 20, 30, 40, 50, 60, 70, 80, 90, 100 or more samples may be collected, for example, by swabbing, from a surface of the one or more surfaces, and suitable ranges may be selected from any of these values, for example 1 to 100, 1 to 50, 1 to 20, 1 to 10, 1 to 5, 10 to 100, 10 to 50, 10 to 20, 20 to 100 or 20 to 50 samples.

When multiple samples are collected from a surface, the average amount of a contaminant across all of the samples collected from the surface may be determined. This average value may then be transmitted to the electronic memory as the contaminant status information corresponding to the surface.

In various embodiments some of the samples collected from a surface may yield a contaminant amount that is deemed to be a statistical outlier. Statistical outliers may be ascribed to, for example, incorrect sampling or equipment malfunction. In various embodiments statistical outliers may be excluded from the average value transmitted to the electronic memory as the contaminant status information corresponding to the surface.

As described herein, in various embodiments information other than location information or contamination status information may also be collected from a surface of the one or more surfaces of a site. Such metadata may be collected at the same time, before or after the collection of the location information and contamination status information.

The contaminant status information, and optionally metadata associated with the surface, the electronic representation of the site is modified to associate the location information, contaminant status information, and optionally the metadata, of the corresponding surface (step C of FIG. 1). In various embodiments modifying the electronic representation comprises updating the representation to display on the representation the contamination status information of the corresponding surface. The contamination status information, when displayed on the electronic representation, appears at the location corresponding to location of the surface from which the contamination status information was collected.

Contamination status information may be displayed on the electronic representation in quantitative terms or qualitative terms. That is, the electronic representation may indicate that a surface is contaminated—a qualitative depiction. Alternatively, the electronic representation may indicate both that a surface is contaminated (the qualitative depiction) and the level of contamination at the surface—a quantitative depiction. Methods of indicating the level of contamination will be apparent to a person skilled in the art. Such methods may include, for example, displaying a bacterial count against a particular surface in the representation or using a key, for example, a series of symbols with each symbol corresponding to a different level of contamination, or a traffic light system with each colour corresponding to a different level of contamination.

The key for the qualitative depiction may be defined by the site manager. Alternatively, the key may be used to indicate acceptable, moderate and unacceptable levels of contamination as defined by industry bodies and/or food safety standards. In other words, a site manager, industry body or food safety standard may define the level of contamination to be allocated to each symbol in a series of symbols or each colour in the traffic light system.

In various embodiments the method comprises repeating the receiving and modifying steps to generate a data set comprising a plurality of associated contamination status information and location information (step D of FIG. 1). The receiving and modifying steps may be repeated as many number of times as desired. For example, the receiving and modifying steps may be repeated at least about 1, 2, 3, 4, 5, 6, 7, 8, 9, 10, 15, 20, 30, 40, 50, 60, 70, 80, 90, 100, 1000, 10,000 or more times, and suitable ranges may be selected from any of these values, for example 1 to 10,000, 1 to 1000, 1 to 100, 1 to 50, 1 to 20, 1 to 10, 1 to 5, 5 to 10,000, 5 to 1000, 5 to 100, 5 to 50, 5 to 20, 5 to 10, 10 to 10,000, 10 to 1000, 10 to 100, 10 to 50, 10 to 20, 20 to 10,000, 20 to 1000, 20 to 100, 20 to 50, 50 to 10,000, 50 to 1000, 50 to 100, 50 to 10,000, 50 to 1000, 50 to 100, 100 to 10,000, 100 to 1000, 1000 to 10,000.

In various embodiments the method may comprise the continuous generation of data sets comprising a plurality of associated contamination status information and location information. In various embodiments the electronic representation may be continuously updated, for example in real time, with the plurality of associated contamination status information and location information. In such embodiments the receiving and modifying steps may be repeated indefinitely, such that the electronic representation is continuously updated, preferably in real time.

Following step D, the data sets are analysed to attribute the source of a contaminant to at or near a surface of the one or more surfaces in step E of FIG. 1. The concept of source attribution as described herein comprises correlating contamination status information with location information. In various embodiments, the concept of source attribution as described herein also comprises analysis of the plurality of contamination status information and the location information to attribute the source of the contaminant to at or near a surface of the one or more surfaces. That is, the method may allow for the original source or origin of the contaminant within a site to be identified.

Analysis of data sets to attribute the source of a contaminant to at or near a surface of the one or more surfaces of a site may be carried out manually or automatically. Manual source attribution may involve analysis of the data sets by a user. The user may then form hypotheses or conclusions about the original source of a contaminant based on patterns in associated location and contamination status information in the electronic representation. Alternatively, software may be used to analyse data sets and attribute the source of a contaminant to at or near a surface of the one or more surfaces of a site.

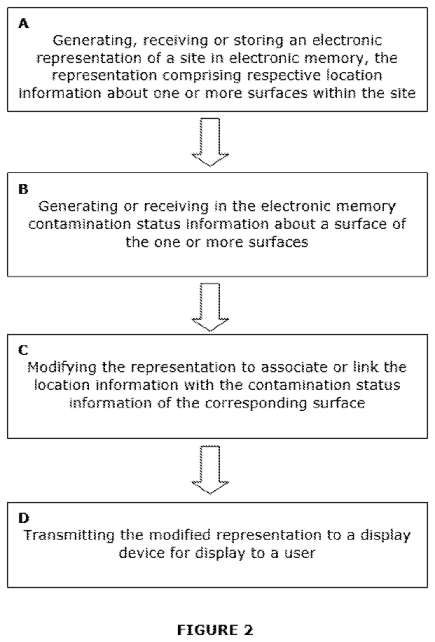

FIG. 2 shows a method according to a second aspect of the invention. FIG. 2 shows the steps in a method for source attribution of a contaminant at a site and displaying a representation thereof, the method comprising
A. generating, receiving or storing an electronic representation of the site in electronic memory, the representation comprising respective location information about one or more surfaces within the site,
B. generating or receiving in the electronic memory contamination status information about a surface of the one or more surfaces,
C. modifying the representation to associate or link the location information with the contamination status information of the corresponding surface, and
D. transmitting the modified representation to a display device for display to a user.

As shown in step A of FIG. 2 an electronic representation of a site may be generated, received or stored in electronic memory. In various embodiments the method may comprise generating and storing an electronic representation in electronic memory. In other embodiments the method may comprise receiving and storing an electronic representation in electronic memory.

Generating an electronic representation may comprise generating one or more than point cloud images or models of a site as described herein. As described herein the one or more point cloud images may be generated using photographs, videos and/or scans of a site, for example scans obtained using 3D laser scanning technologies. As also described herein, the one or more point cloud images or models may comprise one or more internal point cloud images or models and/or one or more external point cloud images or models. Methods of generating internal and external point cloud models have been described herein with reference to FIG. 1 and it will be understood by a person skilled in the art that such methods are also applicable to the method of FIG. 2.

In various embodiments the electronic representation may be an initial representation of a site. That is, the electronic representation may comprise respective location information about one or more surfaces within a site. In various embodiments the initial representation of a site may not comprise contaminant status information. The initial representation may comprise a plan of the site, a 3D model of a site, or one or more images of a site, or any combination of any two or more thereof. The initial representation may be stored in a storage medium as described herein. In various embodiments the initial representation may be stored in a database, for example a database stored in a storage medium as described herein.

An electronic representation may be generated by the company, co-operative or individual carrying out a method in accordance with the invention described herein. Alternatively, in various embodiments the electronic representation may be generated by a third party. In such embodiments, the third party may provide other companies, co-operatives or individuals access to the electronic representation such that the electronic representation is received by the company, co-operative or individual carrying out a method in accordance with the invention.

The company, co-operative or individual carrying out the method of the invention may store one or more copies of the electronic representation or initial representation in electronic memory. When multiple copies of the electronic representation or initial representation are stored in electronic memory, each copy may be stored on the same or on different computers. In various embodiments all or some of the copies may be stored in a network such that a change in one copy is made across all copies within the network.

As shown in step B of FIG. 2, the method may comprise generating or receiving in electronic memory contamination status information about a surface.

The process of generating a contamination status information may comprise converting a raw (quantitative) measurement of a contaminant in a sample into a qualitative depiction to be displayed to a user. For example, as described herein, the contaminant may be bacteria. The raw (quantitative) measurements of a contaminant may comprise a bacterial count expressed in terms of colony forming units (cfu) in a sample taken at a surface of the one or more surfaces of a site. In contrast, a qualitative depiction may comprise a key, for example, a series of symbols corresponding to different levels of contamination or a traffic light system corresponding to different levels of contamination as described herein.

In various embodiments the raw (quantitative) measurement of a contaminant, for example the bacterial count in a sample, may be displayed on an electronic representation. Alternatively, the raw (quantitative) measurement may be converted to a qualitative depiction and the qualitative depiction may then be displayed on an electronic representation.

As described herein with reference to FIG. 1, the key for the qualitative depiction may be defined by the site manager. Alternatively, the key may be used to indicate various levels of contamination as defined by industry bodies and/or food safety standards. For example, an industry body may prescribe that a bacterial count for a hygiene indicator organism might be
below 10² cfu represents an acceptable level of contamination and are to correspond to the colour green in a key using a traffic light system,
between 10² to 10⁵ cfu represents a moderate level of contamination and are to correspond to the colour yellow in a key using a traffic light system, and
above 10⁵ cfu represents an unacceptable level of contamination and are to correspond to the colour red in a key using a traffic light system.

It will be understood by a person skilled in the art that the key for the qualitative depiction may comprise any number of levels. For example, in various embodiments the key may comprise at least about 1, 2, 3, 4, 5, 6, 7, 8, 9, 10 or more levels, and suitable ranges may be selected from any of these values, for example from 1 to 10, 2 to 10, 3 to 10, 4 to 10, 5 to 10, 6 to 10, 7 to 10, 8 to 10, 9 to 10, 1 to 9, 1 to 8, 1 to 7, 1 to 6, 1 to 5, 1 to 4, 1 to 3, 1 to 2, 2 to 8, 2 to 6, or 2 to 4 levels.

In various embodiments contamination status information may be depicted in ways other than using a key comprising symbols or colours. For example, as described herein in various embodiments the respective location information about one or more surfaces within a site may be displayed using a shape corresponding to the surface. The shape may be a 2D or 3D shape. For example, in various embodiments the electronic representation may comprise 3D spheres, each 3D sphere corresponding to a surface within the site. In such embodiments the shape, for example the 3D sphere, may have a size that is proportional to contamination status of the surface.

In various embodiments the generation of contamination status information may be carried out by a third party. That is, the generation of contamination status information may be carried out by a party other than company, co-operative or individual carrying out the method of the invention. In such embodiments the third party may provide other companies, co-operatives or individuals access to the contamination status information such that the contamination status information is received by the company, co-operative or individual carrying out a method in accordance with the invention. The third party may be a company that deals with pest or microbial control, contamination and/or management.

Once contamination status information is generated or received in electronic memory, the electronic representation is modified to associate or link the location information with the contamination status information of the corresponding surface (step C in FIG. 2). As described herein, in various embodiments modifying the electronic representation comprises updating the representation to display on the representation the contamination status information of the corresponding surface. The contamination status information, when displayed on the electronic representation, appears at the location corresponding to location of the surface from which the contamination status information was collected. The modified representation is then transmitted to a display device for display to a user (step D in FIG. 2).

The modified representation may be displayed on any suitable display device, for example any suitable general-purpose computer system or computing device, including, but not limited to, a desktop, laptop, notebook, tablet, smart television, game console or mobile device as described herein. Preferably the modified representation is displayed on a desktop, laptop, notebook, tablet, smart television, or mobile device.

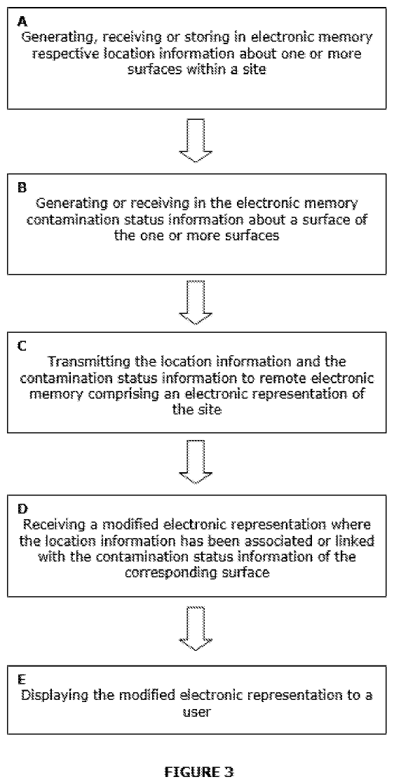

FIG. 3 shows a further method according to an aspect of the invention. FIG. 3 shows the steps in a method for source attribution of a contaminant at a site and displaying a representation thereof, the method comprising
A. generating, receiving or storing in electronic memory respective location information about one or more surfaces within the site,
B. generating or receiving in the electronic memory contamination status information about a surface of the one or more surfaces,
C. transmitting the location information and the contamination status information to remote electronic memory comprising an electronic representation of the site,
D. receiving a modified electronic representation where the location information has been associated or linked with the contamination status information of the corresponding surface, and
E. displaying the modified electronic representation to a user.

Examples of methods for the collection of location information have been described in detail with reference to FIG. 1 and are also applicable to the method depicted in FIG. 3.

As shown in step A of FIG. 3, location information about one or more surfaces of a site may be generated, received or stored. In various embodiments the method may comprise generating and storing location information about one or more surfaces of a site. In other embodiments the method may comprise receiving and storing location information about one or more surfaces of a site.

Generating location information may comprise collecting location information as described herein. Location information may be generated by the company, co-operative or individual carrying out a method in accordance with the invention described herein. For example, as described above, location information may be collected by a person collecting the one or more samples from a surface, or by a different person. Alternatively, in various embodiments the location information may be generated by a third party. In such embodiments, the third party may provide other companies, co-operatives or individuals access to the location information such that the location information is received by the company, co-operative or individual carrying out a method in accordance with the invention. In various embodiments the location information may be stored in electronic memory.

In various embodiments the method comprises generating or receiving in the electronic memory contamination status information about a surface of the one or more surfaces (see step B of FIG. 3). In various embodiments location information and contamination status information may be transmitted to remote electronic memory comprising an electronic representation of the site (see step C of FIG. 3). Methods of generating and receiving location information and/or contamination status information and methods of generating an electronic representation of a site have been described herein in detail with reference to FIG. 1 or 2. Such description is also applicable to the method of FIG. 3.

The transmission of location information and contamination status information to remote electronic memory will depend on factors such as, for example, how the information was collected, who collected the information and how the information is stored. In various embodiments transmission of location information and/or status information may comprise transmission from a remote storage facility, for example a cloud-based storage facility or the internet, to the device used to carry out a computer implemented method of source attribution in accordance with the invention. In various embodiments transmission of location information and/or status information may comprise transmission from a first device on which the information was stored to a second the device. In such embodiments the second device may be the device used to carry out a computer implemented method in accordance with the invention. Transmission may be by any suitable protocol and over any suitable medium, including combinations of protocols and mediums, wired or wireless. Examples of wireless transmission may include one or more of Wifi, Bluetooth, radio frequency (RF), infrared, and the like. Transmission may over one or any combination of networks, including a local area network, a wide area network, a cellular network, an intranet, an internet, the internet, and the like.

As described above with reference to FIGS. 1 and 2, in various embodiments the location information and the contamination status information are associated or linked. The electronic representation of a site may be updated based on the associated or linked location information and contamination status information to generate a modified electronic representation of the site. The modified electronic representation may be generated by the company, co-operative or individual carrying out a method in accordance with the invention. Alternatively, the modified electronic representation may be generated by a third party. That is, a party that does not carry out a method in accordance with the invention.

The electronic representation and modified electronic representation of the site may be generated on the same or on different devices, for example different computers. In various embodiments the electronic representation and/or the modified electronic representation may be stored locally, for example in-house, on-premises or on local storage hardware; or remotely, for example in a cloud-based storage facility or on the internet. For example, the electronic representation and modified electronic representation may be generated on the same device and stored locally on that device.

In various embodiments the electronic representation and/or the modified electronic representation may be generated on the same or different devices and the electronic representation and the modified electronic representation may be stored on a storage medium as described herein. In various embodiments the electronic representation and/or the modified electronic representation may be stored remotely, for example in a cloud-based storage facility or on the internet.

In various embodiments one or more of either or both of the generating steps and the transmitting step are carried out using a point of use hardware device, such as a handheld device. The point of use hardware device may be, for example a notebook, tablet or mobile device, preferably a tablet or a mobile device.

In various embodiments the modified electronic representation may be received, for example from a storage medium, on a device used to implement a computer implemented method of source attribution in accordance with the invention (see step D in FIG. 3).

In various embodiments the modified electronic representation may be displayed to a user. As described herein, the modified representation may be displayed on any suitable display device, for example any suitable general-purpose computer system or computing device, including, but not limited to, a desktop, laptop, notebook, tablet, smart television, game console or mobile device as described herein. Preferably the modified representation is displayed on a desktop, laptop, notebook, tablet, smart television, or mobile device.

The user may be able to interact with the electronic representation and/or the modified electronic representation. For example, the user may be able to add comments to different parts of the representation. Such comments may come to form part of the metadata associated with a surface of the one or more surfaces against which the comments were made.

The inventors believe that the methods described herein may have the potential to provide a fast and transparent resolution of issues related to source attribution in a number of industries.

In various embodiments the methods described herein may be used to monitor contamination at a site. Contaminant status information and source attribution information generated in accordance with the methods described herein may be used to
provide confidence in the safety and quality of products from a site, for example food products from a food preparation site,
provide evidence relating to the safety and quality of products from a site to consumers and/or regulators,
positively link or positively discount a link between contaminants found in one location, for example on one surface, and contamination in another location, for example in a product,
monitor contamination events at a site over a period of time to build a history of events at the site,
provide data for modelling future contamination events at a particular site and/or at particular surfaces within a site, and/or
implement new management activities or additional safety measures, such as for example targeted cleaning, for a site and/or surfaces within a site that are shown to have a higher potential for contamination based on a history of events.

It will be understood by a person skilled in the art that various embodiments or features of the invention, for example the nature of the site, contaminant, surface, methods of generating, receiving or storing an electronic representation, methods of mapping, methods of collecting contamination status information and of displaying such information on the electronic representation described with reference to any one of FIGS. 1-3 may also be applicable to the methods depicted any one of the other Figures.

### EXAMPLES

### Example 1

The described process is used to operate a hygiene management plan at a site where contamination must be closely controlled for protection of human or animal health. A computer-based 2D floorplan map of the site is derived from building plans and a photographic survey and all surfaces of interest relating to possible contamination, such as microbial contamination, are represented on the map. Surfaces of interest include process equipment, ingredient, raw material, component, and packaging ingress points, process, packing and product egress points, cleaning equipment, hygiene control points, drains and other services, points of personnel ingress, movement, congregation, and egress, and personnel touch points including tools, handles and computer and control interaction points.

The plan includes a predefined weekly schedule that specifies location, surface and sampling type information to be collected for each surface, for each contaminant of interest, such as a microbial contaminant. The weekly schedule is created with consideration of likely contamination points, such as microbes' preferred niches informed by the literature, by previous detections, and also includes randomly selected surfaces within the site.

Daily schedules of sampling task sheets are created from the weekly schedule and include all the sampling tasks required to complete the sampling schedule for that day. The daily schedules are deployed as checklists in paper and/or electronic format for operators to take into the site to guide and record sampling.

Samples for analysis, such as microbiological analysis, are taken according to standard operating procedures and sent to a testing location for analysis. Depending on the microbe of interest, specific sampling kits, such as swabs and point of use swabs, and point of use testing apparatus are used. Contaminant identification analysis is conducted using rapid tests, such as rapid PCR based tests using primers with specificity to microbes of interest and following the standard operating procedure for the test.

The results of testing, both positive and negative detections, are recorded by overlaying respective icons and links to extended sampling and test information onto surfaces represented on the map, such that the map is displaying a representation of the ongoing swab and test results occurring within the site and continually updated as results are obtained.

The map and overlaid results are analysed periodically, for example weekly. The accumulation of test results over a long period, for example a period of time relevant to the site, such as a shift, a week, a month, or a production season to date, is analysed to highlight areas of higher and lower incidence respectively, for each contaminant of interest. Results are indexed to activities within the site, where possible, for example product scheduling or maintenance activities, to reveal patterns of activities against test results.

A remedial management plan is created including actions such as specific cleaning programmes, maintenance tasks or increased testing surveillance, taking into account the incidence rates and contamination risk posed by each surface and location.

### Example 2

In response to a contamination event, such as microbial contamination, at a site where contamination must be closely controlled for protection of human or animal health, the described process is used to locate the source, trace the movement from the source, and create remedial management actions. A computer-based representation of the site is created as a point cloud model of the facility compiled from multiple 3D laser scans using the appropriate scanner's software. All of the surfaces within the 3D model are considered candidates for sampling.

Using the 3D model as a guide, a surface sampling plan is created taking into account known niches for the contaminants, such as microbes, of interest, routes of vectors such as ingredients, product, services and personnel, knowledge of previous detection locations, and using statistical sampling considerations such as randomised grid based sample selection and random selection from otherwise equivalent surfaces. The number of surfaces to be sampled is scaled according to the site. Sampling task sheets are created from the sampling plans and include all the sampling tasks required to complete the sampling plan and deployed as checklists in paper and/or portable electronic format for operators to take into site to guide and record sampling.

A schedule of sampling is created, where the sampling described in the sampling plan is repeated after set time intervals, for example 1 month later, or after a prescribed intervention. The sampling plan site is actioned.

All samples are directly moved to the test location after sampling. Historical samples, if available, are added to the sample pool to further increase the time span of the studied samples. The initial testing is to isolate contaminants of interest, such as microbes of interest from background microflora using specific enrichment techniques for that contaminant.

In the case of microbial contaminants, candidate colonies from each enrichment step are analysed using MALDI-TOF-MS type microbial identification apparatus to identify to at least the genus level. DNA from the selected colonies of interest are extracted using a standard laboratory process, checked for adherence to quality metrics, and then submitted to next generation whole genome sequencing and a bioinformatics pipeline to at least de-complex, trim, clean and assemble into FASTA format genome sequences in preparation for subsequent analysis. Sequences are analysed with (1) WGS based species identification software, to confirm identity of the species, strain and subtype(s) if applicable, and (2) SNP analysis where a sample's genome sequence is compared to a closely related reference genome of the same species, and the number of differences is tallied. The output from SNP analysis is used to create a table of the relatedness (closeness) between isolates based on the number of SNP's between each sample and each and every other sample. Using clustering analysis, the SNP data is used to determine if the population of species of interest is comprised of single or multiple clusters of similar isolates, and whether sub-populations appear to be transitory, single or multiple incursion, or incumbent within the facility. A known molecular evolutionary time period for SNP changes within the species of interest is used to define clonality, and overlaying the closeness of SNPs on the map of the facility is used to trace pathways of clonal isolates in 3D through the site.

The combination of scheduling repeated sampling exercises over long periods of time and connecting samples based on relatedness and by surface locations allows epidemiological considerations to be then applied to locate the source, transport mechanism, and potential product contamination routes of the species of interest. A database comprising microorganism identifiers and associated contamination risk or hazard information is used to associate a risk relating to the surface and potential microorganism contaminant.

The 3D mapping of all results and risk scores allows rapid communication of the outcomes in 3D format. Remedial management actions are considered, for example maintenance, changes to surfaces, altering vector flows, changes to cleaning schedules, to remove the contamination, address the source and/or prevent movement of the species of interest, and a further scheduled surface swabbing exercise is used to evaluate the effectiveness of the management intervention. The data collected is re-used to add to or create a routine hygiene plan as exemplified in Example 1.

The foregoing description of the invention includes preferred forms thereof. Modifications may be made thereto without departing from the scope of the invention as defined by the accompanying claims.

1. A computer-implemented method for source attribution of a contaminant at a site, the method comprising
storing an electronic representation of the site in electronic memory, the representation comprising respective location information about one or more surfaces within the site,
receiving in the electronic memory contamination status information about a surface of the one or more surfaces,
modifying the representation to associate the location information with the contamination status information of the corresponding surface,
repeating the receiving and modifying steps to generate a data set comprising a plurality of associated contamination status information and location information, and
analysing the data set to attribute the source of the contaminant to at or near a surface of the one or more surfaces,
wherein the contamination status information comprises nucleic acid sequence information, is generated according to a schedule, and is determined by a method comprising collecting two or more samples from the one or more surfaces according to the schedule and analysing the two or more samples to determine the relatedness of the two or more samples.

2. A computer-implemented method for source attribution of a contaminant at a site and displaying a representation thereof, the method comprising
generating, receiving or storing an electronic representation of the site in electronic memory, the representation comprising respective location information about one or more surfaces within the site,
generating or receiving in the electronic memory contamination status information about a surface of the one or more surfaces,
modifying the representation to associate or link the location information with the contamination status information of the corresponding surface, and
transmitting the modified representation to a display device for display to a user,
wherein the contamination status information comprises nucleic acid sequence information, is generated according to a schedule, and is determined by a method comprising collecting two or more samples from the one or more surfaces according to the schedule and analysing the two or more samples to determine the relatedness of the two or more samples.

3. The computer-implemented method of claim 2, further comprising generating the representation by a method comprising
generating or receiving an initial representation of the site comprising a digital plan of the site, a 3D digital model of the site, or one or more images of the site, or any combination of any two or more thereof,
generating or receiving location information about respective one or more surfaces within the site,
storing the initial representation and the location information in a database, and
associating or linking the initial representation with the location information, such that a surface of the one or more surfaces can be identified by its respective location information.

4. A computer-implemented method for source attribution of a contaminant at a site and displaying a representation thereof, the method comprising
generating, receiving or storing in electronic memory respective location information about one or more surfaces within the site,
generating or receiving in the electronic memory contamination status information about a surface of the one or more surfaces,
transmitting the location information and the contamination status information to remote electronic memory comprising an electronic representation of the site,
receiving a modified electronic representation where the location information has been associated or linked with the contamination status information of the corresponding surface, and
displaying the modified electronic representation to a user,
wherein the contamination status information comprises nucleic acid sequence information, is generated according to a schedule, and is determined by a method comprising collecting two or more samples from the one or more surfaces according to the schedule and analysing the two or more samples to determine the relatedness of the two or more samples.

5. The computer-implemented method of claim 4, wherein one or more of either or both of the generating steps and the transmitting step are carried out using a point of use hardware device.

6. The computer-implemented method of any one of claims 1 to 5, wherein the contamination status information further comprises amino acid sequence information, microbiological assay information, chemical assay information, or biochemical assay information, or any combination of any two or more thereof.

7. The computer-implemented method of claim 6, wherein the nucleic acid sequence information comprises one or more partial or whole genome sequences or mixed genome sequences.

8. The computer-implemented method of claim 6 or 7, wherein the nucleic acid sequence information comprises ribosomal RNA sequence information, single nucleotide polymorphism (SNPs) information or metagenomic information.

9. The computer-implemented method of any one of claims 7 to 9, wherein the nucleic acid sequence information is generated using nucleic acid sequencing methods including but not limited to Sanger sequencing, whole genome sequencing (WGS), or next generation sequencing (NGS).

10. The computer-implemented method of claim 6, wherein the microbiological assay information comprises mass spectrometry information.

11. The computer-implemented method of any one of claims 1 to 10, wherein the site is a food production site, a food handling site, a food preparation site, a food logistics site, a food consumption site, an agricultural site, an animal handling site, a medical facility, a building, a vehicle, or a structure.

12. The computer-implemented method of any one of claims 6 to 11, wherein the contamination status information comprises information about a nucleic acid containing material, including but not limited to a bacteria, a virus, a protozoa, a plant, or an animal, or any combination of any two or more thereof.

13. The computer-implemented method of any one of claims 1 to 12, wherein the representation comprises a 3D digital model, a point cloud, or one or more images of the site, or any combination thereof.

14. The computer-implemented method of claim 13, wherein the point cloud is generated by photogrammetrically processing the one or more images of the site.

15. The computer-implemented method of claim 13, wherein the point cloud is generated by laser, radar or sonar distance measurement of the site.

16. The computer-implemented method of any one of claims 1 to 15, wherein the representation comprises unique identifiers linked to the one or more surfaces.

17. The computer-implemented method of any one of claims 1 to 16, wherein the contamination status information is determined by a method comprising collecting one or more samples from the one or more surfaces and analysing the one or more samples.

18. The computer-implemented method of claim 17, wherein the one or more samples are obtained according to a sampling plan.

19. The computer-implemented method of claim 18, wherein the sampling plan provides a first sampling location, a second sampling location, and an n^{th} sampling location, wherein each sampling location is determined by one or more statistically-based sampling methods.

20. The computer-implemented method of claim 19, wherein the statistically-based method is simple random sampling, the method comprising selecting within the site a first random sampling location, a second random sampling location, and an n^{th} random sampling location.

21. The computer-implemented method of claim 19, wherein the statistically-based method is systematic sampling, the method comprising defining a grid within the site, selecting a first sampling location on the grid, selecting a second sampling location on the grid, and selecting an n^{th} random location on the grid.

22. The computer-implemented method of claim 18, wherein the statistically-based method is adaptive cluster sampling, the method comprising randomly selecting within the site a first set of primary sampling locations, determining whether each primary sampling location contains a contaminant, and selecting a second set of secondary sampling locations, each secondary sampling location being adjacent to a primary sampling location that contains a contaminant.

23. The computer-implemented method of claims 17 to 22, wherein the one or more samples are collected by swabbing, wiping, vacuuming, or blotting or any combination thereof.

24. The computer-implemented method of claim 17, wherein analysis of the one or more samples comprises determining the presence of microbial contaminants using an assay to identify nucleic acid or amino acid information, or a microbiological assay, a chemical assay, or a biochemical assay, or any combination thereof.

25. The computer-implemented method of any one of claims 17 to 24, wherein analysis of the one or more samples comprises determining the identity of a contaminant.

26. The computer-implemented method of any one of claims 17 to 25, wherein the analysis of the one or more samples comprises determining the relatedness of two or more samples.

27. The computer-implemented method of any one of claims 17 to 26, wherein the analysis of the one or more samples comprises determining the movement of a contaminant within a site by a method comprising
obtaining assay information from each of two or more samples obtained from different locations within the site,
determining the relatedness of the samples by analysing the assay information,
identifying potential vectors of contamination, and
determining the movement of the contaminant within the site by comparing the relatedness of the samples with the potential vectors.

28. The computer-implemented method of any one of claims 17 to 24, wherein analysis of the one or more samples comprises determining the identity of a microorganism contaminant by a method comprising
obtaining a first nucleic acid sequence from the one or more samples,
providing a second nucleic acid sequence from a reference microorganism,
defining a threshold of sequence similarity for establishing the identity of a microorganism in the one or more samples, and
determining whether the sequence similarity between the first and second nucleic acid sequences meets the threshold.

29. The computer-implemented method of claim 28, wherein the threshold for establishing identity is a sequence similarity of at least about 60, 70, 80, 90, 95, or 99 percent.

30. The computer-implemented method of claim 28 or claim 29, wherein the method is repeated for 10, 100, or 100 or more nucleic acid sequences from the one or more samples.

31. The computer-implemented method of any one of claims 28 to 30, wherein the identity of the microorganism comprises genus, species and/or strain information.

32. The computer-implemented method of any one of claims 28 to 31, further comprising determining a level of risk associated with the microorganism contaminant by comparing the identity of the microorganism contaminant to a database comprising microorganism identifiers and associated risk or hazard information, and associating a risk or hazard to the microorganism contaminant.

33. The computer-implemented method of any one of claims 17 to 24 and 28 to 32, wherein the analysis of the one or more samples comprises determining the relatedness of two or more samples by a method comprising
obtaining a first nucleic acid sequence from a first sample,
obtaining a second nucleic acid sequence from a second sample or from a reference source, and
comparing the first and second nucleic acid sequences to determine the relatedness of the samples.

34. The computer-implemented method of claim 33, wherein comparing the first and second nucleic acid sequences to determine the relatedness of the samples comprises
identifying one or more SNPs between each of the first and second nucleic acid sequences, and
comparing the SNPs from the first and second nucleic acid sequences to determine the relatedness of the samples.

35. The computer-implemented method of any one of claims 17 to 24 and 28 to 34, wherein the analysis of the one or more samples comprises determining the movement of a microorganism contaminant within a site by a method comprising
obtaining nucleic acid sequence information from each of two or more samples obtained from different locations within the site,
determining the relatedness of the samples by analysing the nucleic acid sequence information,
identifying potential vectors of contamination, and
determining the movement of the microorganism contaminant within the site by comparing the relatedness of the samples with the potential vectors.
